SEQUENCE LISTING (22 sequences)

SEQ ID NO: 1 - DNA, 27 bp, Human adenovirus type 3 E3/19k
cgaattcaacatccaagatgaaggtac

SEQ ID NO: 2 - DNA, 46 bp, Human adenovirus type 3 E3/19k
cggaattctcagtgatggtgatggtgatgaggcattttctttcatc

SEQ ID NO: 3 - DNA, 26 bp, human cytomegalovirus US2
gcggatccacacgctgtttcaccatg

SEQ ID NO: 4 - DNA, 27 bp, human cytomegalovirus US2
gcgaattcccgggcgtctcagcacacg

SEQ ID NO: 5 - DNA, 29 bp, human cytomegalovirus US3
gcggatccttcggagccatgaagccggtg

SEQ ID NO: 6 - DNA, 28 bp, human cytomegalovirus US3
gcggaattcgtacctgttaaataaatcg

SEQ ID NO: 7 - DNA, 26 bp, human cytomegalovirus US6
gcggatccttcactatggatctcttg

SEQ ID NO: 8 - DNA, 30 bp, human cytomegalovirus US6
cgaattcatcaggagccacaacgtcgaatc

SEQ ID NO: 9 - DNA, 27 bp, human cytomegalovirus US11
gcggatccttgtaagacagaatgaacc

SEQ ID NO: 10 - DNA, 28 bp, human cytomegalovirus US11
gcgaattcagttctatatatcaccactg

SEQ ID NO: 11 - DNA, 27 bp, human herpesvirus 2 ICP47
ccgaattcgagatcgtatcaaggggcc

SEQ ID NO: 12 - DNA, 26 bp, human herpesvirus 2 ICP47
ccggatccgggacaccatgtcttggg

SEQ ID NO: 13 - DNA, 25 bp, human B7.1
ctaagcttctgaagccatgggccac

SEQ ID NO: 14 - DNA, 29 bp, human B7.1
ggctcgagctgcggacactgttatacagg

SEQ ID NO: 15 - DNA, 600 bp, human cytomegalovirus Towne strain US2
atgaacaatctctggaaagcctgggtgggtctttggacctccatgggtcccttgatccgcctgcccgatggcatcactaaagccggggaagacgcgctccggccctggaagtccacggccaagcatccctggtttcaaatcgaggacaaccggtgctacattgacaacggcaagttgtttgctcgggggagcatcgtgggcaacatgagtcggttcgtcttcgatccgaaggccgattatggcggcgtgggagagaacctgtacgtacacgcggacgacgtggagttcgttcccggggagtcgttaaagtggaacgtgcggaacttagatgtgatgccgatcttcgagacgctagccctgcgtctggtactgcaaggggatgtgatctggctgcgttgcgtccccgaactgcgagtagactacacgtctagcgcgtacatgtggaacatgcagtacgggatggtgcggaagtcatacacgcatgtggcctggacaatagtgttttactccataaacattaccctgttggtattgtttatcgtgtatgtgactgtggactgtaacttgtctatgatgtggatgcggtttttcgtgtgctga

SEQ ID NO: 16 - DNA, 561 bp, human cytomegalovirus Towne strain US3
atgaagccggtgttggtgctcgcgatcctggccgtcttgttcctgagactcgcggactcggtgccccggcccctggatgtggtggtatcggagatcagatcggcccactttcgggtggaggagaaccaatgctggtcccatatgggcatgctacactacaaggggaggatgtcgggcaacttcaccgagaaacactttgtgagcgtgggtatcgtctctcaatcttacatggacagactgcaggtgtccggtgagcagtaccaccatgatgagcgcggagcctatttcgagtggaacatcggtgggcacccggtgccgcacaccgtggatatggtggacatcacgttgtctacgaggtggggagaccccaagaaatatgccgcgtgtgttcctcaggtgaggatggactatagctctcataccatcaactggtaccttcagcgaagcataagagacgacaattggggtctgctgttcagaaccctgctcgcatatctgttctctctggttggtcttgtgctcctgaccgtaggggtgagcgcccgtctgccatttatttaa

SEQ ID NO: 17 - DNA, 552 bp, human cytomegalovirus Towne strain US6
atggatctcttgattcgtctcggttttctgttgatgtgtgcgttgccgacccccggtgagcggtcttcgcgtgacccgaaaacccttctctctctgtctccgcgacaacaagcttgtgttccgagaacgaagtcgcacagacccgtttgttacaacgatacaggggactgcacagatgcagatgatagctggaaacagctgggtgaggactttgcgcaccaatgcttgcaggcggcgaaaaagaggcctaaaacgcacaaatcccgtccgaacgataggaaccttgagggtaggctgacctgtcaacgagtccgtcggctactgccctgtgatttggatattcatcctagccaccggttgttaacgcttatgaataactgcgtctgtgacggggccgtttggaacgcgtttcgcttgatagaacgacacggattcttcgctgtgactttgtatttatgttgcgggattactctgctggttgttattctagcattgctgtgcagcataacatacgaatcgactggacgtgggattcgacgttgtggctcctga

SEQ ID NO: 18 - DNA, 648 bp, human cytomegalovirus Towne strain US11
atgaaccttataatgcttattctagccctctgggccccggtcgcgggtagtatgcctgaattatccttgactcttttcgatgaacctccgcccttggtggagacggaaccgttaccgcctctgcccgatgtttcggagtaccgagtagagtattccgaggcgcgctgcgtgctccgatcgggcggtcgactggaggctctgtggaccctgcgcgggaacctgtccgtgcccacgccgacaccccgggtgtactaccagacgctggagggctacgcggatcgagtgccgaccccggtggaggacatctccgaaagccttgtcgcaaaacgctactggctccgggactatcgtgttccccaacgcacaaaactcgtgttgttctacttttccccctgccatcaatgccaaacttattatgtagagtgcgaaccccggtgcctcgtgccttgggttcccctgtggagctcgttagaggacatcgaacgactattgttcgaagatcgccgtctaatggcgtactacgcgctcacgattaagtcggcgcagtatacgctgatgatggtggcagtgattcaagtgttttgggggctgtatgtgaaaggttggctgcaccgacattttccctggatgttttcggaccagtggtga

SEQ ID NO: 19 - protein, 199 aa, human cytomegalovirus Towne strain US2
MNNLWKAWVGLWTSMGPLIRLPDGITKAGEDALRPWKSTAKHPWFQIEDNRCYIDNGKLFARGSIVGNMSRFVFDPKADYGGVGENLYVHADDVEFVPGESLKWNVRNLDVMPIFETLALRLVLQGDVIWLRCVPELRVDYTSSAYMWNMQYGMVRKSYTHVAWTIVFYSINITLLVLFIVYVTVDCNLSMMWMRFFVC

SEQ ID NO: 20 - protein, 186 aa, human cytomegalovirus Towne strain US3
MKPVLVLAILAVLFLRLADSVPRPLDVVVSEIRSAHFRVEENQCWSHMGMLHYKGRMSGNFTEKHFVSVGIVSQSYMDRLQVSGEQYHHDERGAYFEWNIGGHPVPHTVDMVDITLSTRWGDPKKYAACVPQVRMDYSSHTINWYLQRSIRDDNWGLLFRTLLAYLFSLVGLVLLTVGVSARLPFI

SEQ ID NO: 21 - protein, 183 aa, human cytomegalovirus Towne strain US6
MDLLIRLGFLLMCALPTPGERSSRDPKTLLSLSPRQQACVPRTKSHRPVCYNDTGDCTDADDSWKQLGEDFAHQCLQAAKKRPKTHKSRPNDRNLEGRLTCQRVRRLLPCDLDIHPSHRLLTLMNNCVCDGAVWNAFRLIERHGFFAVTLYLCCGITLLVVILALLCSITYESTGRGIRRCGS

SEQ ID NO: 22 - protein, 215 aa, human cytomegalovirus Towne strain US11
MNLIMLILALWAPVAGSMPELSLTLFDEPPPLVETEPLPPLPDVSEYRVEYSEARCVLRSGGRLEALWTLRGNLSVPTPTPRVYYQTLEGYADRVPTPVEDISESLVAKRYWLRDYRVPQRTKLVLFYFSPCHQCQTYYVECEPRCLVPWVPLWSSLEDIERLLFEDRRLMAYYALTIKSAQYTLMMVAVIQVFWGLYVKGWLHRHFPWMFSDQW

(19) United States
(12) Patent Application Publication
(10) Pub. No.: US 2003/0118568 A1
(43) Pub. Date: Jun. 26, 2003

(54) Viral stealth technology to prevent T cell-mediated rejection of xenografts

(75) Inventor: Mark D. Crew, Little Rock, AR (US)

Correspondence Address: WRIGHT, LINDSEY & JENNINGS LLP, Suite 102, 320 North Rollston Avenue, Fayetteville, AR, 72701, US

(73) Assignee: Board of Trustees of the University of Arkansas

(21) Appl. No.: 10/300,393

(22) Filed: Nov. 19, 2002

Related U.S. Application Data
(60) Provisional application No. 60/342,981, filed on Dec. 18, 2001.

(51) Int. Cl. A01K 67/00; C07H 21/04; A61K 48/00; C12P 21/02; C12N 5/06; C12N 5/08; C07K 14/705

(52) U.S. Cl. 424/93.21; 800/8; 435/69.1; 435/372; 435/320.1; 435/325; 530/350; 536/23.5

(57) ABSTRACT

The invention comprises exploiting viral stealth mechanisms to eliminate pig MHC class I cell-surface expression. PK(15) (pig kidney) cells stably transfected with the Herpes Simplex Virus (HSV) ICP47 gene [PK(15)-ICP47 cells] exhibited a dramatic reduction of MHC class I cell-surface expression when compared to untransfected PK(15) cells. To test the effect of down-regulation of porcine MHC class I on human cellular immune responses, a human CD8+ enriched T cell line (anti-PK15 T cells) with reactivity towards PK(15) cells was derived by repeated stimulation of human T cells with PK(15) cells stably transfected with the co-stimulatory molecule B7.1 [PK(15)-B7.1 cells]. Anti-PK15 T cells efficiently lysed PK(15) cells but not PK(15)-ICP47 (class I negative) cells. Consistent with effector function, anti-PK15 T cells showed a robust proliferative response to PK(15)-B7.1 cells but did not proliferate at all to PK(15)-B7.1 cells which also expressed HSV ICP47.

### CROSS-REFERENCE TO RELATED APPLICATIONS

The present application claims the benefit of U.S. Provisional Application No. 60/342,981, filed Dec. 18, 2001, which is incorporated herein by reference.

### STATEMENT REGARDING FEDERALLY SPONSORED RESEARCH OR DEVELOPMENT

The invention was made with Government support under the terms of Grant No. Al49885 awarded by NIH/NIAID and the Office of Research and Development, Department of Veterans Affairs. The Government has certain rights in the invention.

### BACKGROUND OF THE INVENTION

1. Field of the Invention

The present invention relates to utilizing virus stealth technology to eliminate pig MHC class I cell-surface expression that hinders the pig-to-human xenotransplantation.

2. Brief Description of the Related Art

Hyperacute rejection (HAR) and delayed xenograft rejection (DXR, also termed acute vascular rejection), are major hurdles to successful pig-to-primate xenotransplantation although these phenomena are an almost negligible consideration in allotransplantation (Auchinocloss, H and Sachs DH, Ann Rev Immunol 1998; 16: 433; Cascalho M and Platt J L., Immunity 2001; 14: 437). However, if HAR and DXR can be overcome then xenografts are likely to be rejected by the same mechanisms operative in the rejection of allografts (between HLA-mismatched individuals), that is, in a manner mediated by T cells. In fact, there is ample evidence that human T cells recognize and respond to porcine cells (Dersimonian H, et al., J Immunol 1999; 162: 6993; Xu XC et al., Transplantation 1999; 68: 473; Lalain S, et al., Diabetologia 1999; 42: 330; Yi S, et al., Transplantation 1999; 67: 435; Vallee I, et al., J Immunol 1998; 161: 1652; Brevig T and Kristensen T., Apmis 1997; 105: 290; Yamada K, et al., J Immunol 1995; 155: 5249; Bravery C A et al., Transplantation 1995; 60: 1024) and moreover it is clear that xenoreactive T cells exist at a measurable frequency in “naïve” individuals (Hartig C V et al., J Immunol 2000; 164: 2790).

In theory, human T cells can recognize pig cells either directly, via interaction of human T cell receptor (TCR) with pig major histocompatibility complex (MHC) class I or II antigens, or indirectly, where human TCRs recognize porcine-derived peptide antigens presented by human MHC class I or II proteins on the cell surface of human antigen presenting cells (APCs). In actuality, both direct and indirect recognition of pig cells by human CD4+ and CD8+ T cells have been documented (Dersimonian H, et al., J Immunol 1999; 162: 6993; Xu XC et al., Transplantation 1999; 68: 473; Lalain S, et al., Diabetologia 1999; 42: 330; Yi S, et al., Transplantation 1999; 67: 435; Vallee I, et al., J Immunol 1998; 161: 1652; Brevig T and Kristensen T., Apmis 1997; 105: 290; Yamada K, et al., J Immunol 1995; 155: 5249; Bravery C A et al., Transplantation 1995; 60: 1024; Hartig C V et al., J Immunol 2000; 164: 2790). In many cases, the evidence for direct recognition is demonstrated by using mAbs directed against pig MHC (swine leukocyte antigen, SLA) class I or II proteins (where these mAbs block proliferative or cytotoxic responses) (Dersimonian H, et al., J Immunol 1999; 162: 6993). In other instances, direct antigen presentation via SLA class I or II proteins is shown by omitting human APCs from proliferation and/or cytotoxicity assays (Xu XC et al., Transplantation 1999; 68: 473).

The cell-surface expression of MHC class I proteins is contingent upon their non-covalent association with β2-microglobulin (β2m) and an 8-9 amino acid peptide in the endoplasmic reticulum (ER) lumen (reviewed in Pamer E and Cresswell P, Annu Rev Immunol 1998; 16: 323; York I A and Rock K L, Annu Rev Immunol 1996; 14: 369). These peptides, generated in the cytosol by the multi-catalytic proteosome, are delivered into the ER by the transporter associated with antigen processing (TAP), a member of the ABC transporter family. Following binding of peptide to the MHC class I protein, the heterotrimeric complex is trafficked through the Golgi to the cell-surface. In principle, the destruction of porcine xenografts by human xenoreactive T cells which occurring by direct recognition of SLA class I proteins could be abrogated by eliminating donor (i.e. pig) MHC class I cell-surface expression. Abolishing SLA class I cell-surface expression could be accomplished by perturbing the intra-cellular assembly of SLA class I proteins at any one of the ordered steps in MHC class I biosynthesis.

The immune system recognizes virally infected cells via MHC class I presentation of virally-derived peptides to CD8+ T cells and many viruses have evolved means to escape T cell recognition by interfering with MHC class I biosynthesis. There is a growing list of virally-encoded proteins which diminish MHC class I cell-surface expression (reviewed in Ploegh H L, Science 1998; 280: 248; Fruh K, et al., Immunol Rev 1999; 168:157; Alcami A and Koszinowski U H, Immunol Today; 2000 21: 447). To list just a few: (i) Both the ICP47 proteins encoded by Herpes Simplex Virus (HSV) type I and II and the US6 gene product of human cytomegalovirus (HCMV) each inhibit TAP, albeit by different mechanisms (Ahn K, et al., Immunity 1997; 6: 613; Fruh K, et al., Nature 1995; 375: 415; Hill A, et al., Nature 1995; 375: 411). (ii) The products of the US2 and US11 genes of HCMV dislocate MHC class I proteins from the ER to the cytosol (Wiertz E J et al., Cell 1996; 84: 769). (iii) HCMV US3, in contrast, impairs the export of class I molecules from the ER (Jones T R et al., Proc Natl Acad Sci USA. 1996; 93: 11327). (iv) Lastly, the adenovirus E3/19K protein not only retains MHC class I proteins in the ER but also inhibits TAP function (Bennett E M, et al., J. Immunol 1999; 62: 5049).

Interference of MHC class I biosynthesis gives viruses a certain degree of stealth, that is, an ability to hide from the host immune system. Exploiting virus stealth technology might therefore be useful in xenotransptantation insofar as “hiding” from the human immune system may lead to extended porcine xenograft survival. References mentioned in this background section are not admitted to be prior art with respect to the present invention.

### BRIEF SUMMARY OF THE INVENTION

Porcine xenograft destruction as a consequence of direct recognition of pig MHC proteins by human T cells is likely to be a significant barrier to successful pig-to-primate xenotransplantation. Perhaps the most straightforward solution to this problem is to eliminate the cell-surface expression of donor (pig) MHC proteins. Viruses have a similar problem in that host MHC proteins alert host T cells to their presence. Because of this, it is advantageous to the virus to develop strategies to inhibit host MHC cell-surface expression. Many viruses in fact have independently evolved mechanisms to achieve a certain degree of stealth by expressing genes whose products interfere with MHC class I biosynthesis.

The present invention is directed to utilizing virus stealth technology to eliminate pig MHC class I cell-surface expression that occurs with pig-to-human xenotransplantation. We have exploited viral stealth mechanisms to eliminate pig MHC class I cell-surface expression. Viral gene products which inhibit MHC class I cell-surface expression function as “dominant negatives”. The use of dominant negative inhibitors of MHC class I expression has several advantages compared to the elimination of MHC expression in β2m or TAP gene “knockout” pigs. These include not requiring homozygosity at any locus and the ability to regulate the expression of the dominant negative inhibitor (so that the pigs are not immunocompromised).

PK(15) (pig kidney) cells stably transfected with the Herpes Simplex Virus (HSV) ICP47 gene [PK(15)-ICP47 cells] exhibited a dramatic reduction of MHC class I cell-surface expression when compared to untransfected PK(15) cells. To test the effect of down-regulation of porcine MHC class I on human cellular immune responses, a human CD8+ enriched T cell line (anti-PK15 T cells) with reactivity towards PK(15) cells was derived by repeated stimulation of human T cells with PK(15) cells stably transfected with the co-stimulatory molecule B7.1 [PK(15)-B7.1 cells]. Anti-PK15 T cells efficiently lysed PK(15) cells but not PK(15)-ICP47 (class I negative) cells. Consistent with effector function, anti-PK15 T cells showed a robust proliferative response to PK(15)-B7.1 cells but did not proliferate at all to PK(15)-B7.1 cells which also expressed HSV ICP47.

### BRIEF DESCRIPTION OF THE DRAWINGS

These and other features, objects and advantages of the present invention will become better understood from a consideration of the following detailed description and accompanying drawings.

FIG. 1 shows the lack of down regulation of pig MHC class I cell-surface expression by human CMV (HCMV) and adenovirus genes. FIG. 1A is untransfected PK(15) cells where the open curve represents staining with an isotype control and filled curves represent staining with the PT85A mAb. FIG. 1B is a FACS analysis of PK(15) cells stably transfected with HCMV US6. FIG. 1C is a FACS analysis of PK(15) cells stably transfected with HCMV US2. FIG. 1D is a FACS analysis of PK(15) cells stably transfected with HCMV US11. FIG. 1E is a FACS analysis of PK(15) cells stably transfected with HCMV US3. FIG. 1F is a FACS analysis of PK(15) cells stably transfected with adenovirus E3/19K.

FIG. 2 shows a dramatic reduction of pig MHC class I cell-surface expression by human HSV type II ICP47. FIG. 2A is untransfected PK(15) cells where the open curve represents staining with an isotype control and filled curves represent staining with the PT85A mAb. FIG. 2B is a FACS analysis of PK(15) cells stably transfected with HSV II ICP47, unsorted. FIG. 2C is a FACS analysis of PK(15) cells stably transfected with HSV II ICP47, sorted.

FIG. 3 shows the FACS analysis of PK(15)-B7.1 and -ICP47 transfectants. FIG. 3A shows the FACS analysis of untransfected PK(15) cells. FIG. 3B shows the FACS analysis of PK(15) stably transfected with ICP47 alone (PK(15)-ICP47). FIG. 3C shows the FACS analysis of PK(15) stably transfected with B7.1 alone (PK(15)-B7.1). FIG. 3D shows the FACS analysis of PK(15) stably transfected together (PK(15)-B7.1/ICP47). PK(15) stably transfected with B7.1 or ICP47 alone (PK(15)-B7.1 and, PK(15)-ICP47, respectively) or together were stained with mAbs specific for MHC class I (PT85A) or B7.1 (CD80) conjugated with PE or FITC, respectively. Single gene transfectants were obtained by G-418 selection. PK(15)-B7.1/ICP47 cells were derived from PK(15)-B7.1 cells by co-transfecting an ICP47 expression vector with a puromycin resistance gene and selection in puromycin.

FIG. 4 shows human CD8+ T cell proliferative response to pig epithelial cells [PK(15) cells]. FIG. 4A is human lymphocytes were subjected to two color FACS analysis with anti-CD4-PE and anti-CD8-FITC mAbs prior to stimulation. FIG. 4B is human lymphocytes were subjected to two color FACS analysis with anti-CD4-PE and anti-CD8-FITC mAbs after repeated stimulation with PK(15) cells stably transfected with a human B7.1 (CD80) expression vector.

FIG. 5 shows the reduced human T cell proliferative responses to pig cells expressing ICP47. Fresh (“naïve”) human PBLs (FIG. 5A) or xenoreactive T cells [i.e. repeatedly stimulated with PK(15)-B7.1 cells] (FIG. 5B) from the same donor were used in cell proliferation (³H-thymidine incorporation) assays with irradiated (30,000 rad) PK(15), PK(15)-B7.1, or PK(15)-B7.1/ICP47 cells as stimulators as indicated. Two-day co-culture (at 10:1 effector:stimulator ratio) was followed by a two-day pulse with ³H-thymidine.

FIG. 6 shows ICP47 inhibits T cell-mediated lysis of pig [PK(15)] cells. Standard cytotoxicity (⁵¹Cr release) assays were performed using as targets PK(15) cells or PK(15) cells stably expressing ICP47. Effector cells were anti-PK15 T cells (see FIG. 4) at an effector:target ratio of 10:1. ⁵¹Cr release was measured after 4, 6, 8, or 20 hours of incubation as indicated.

### DETAILED DESCRIPTION OF THE INVENTION

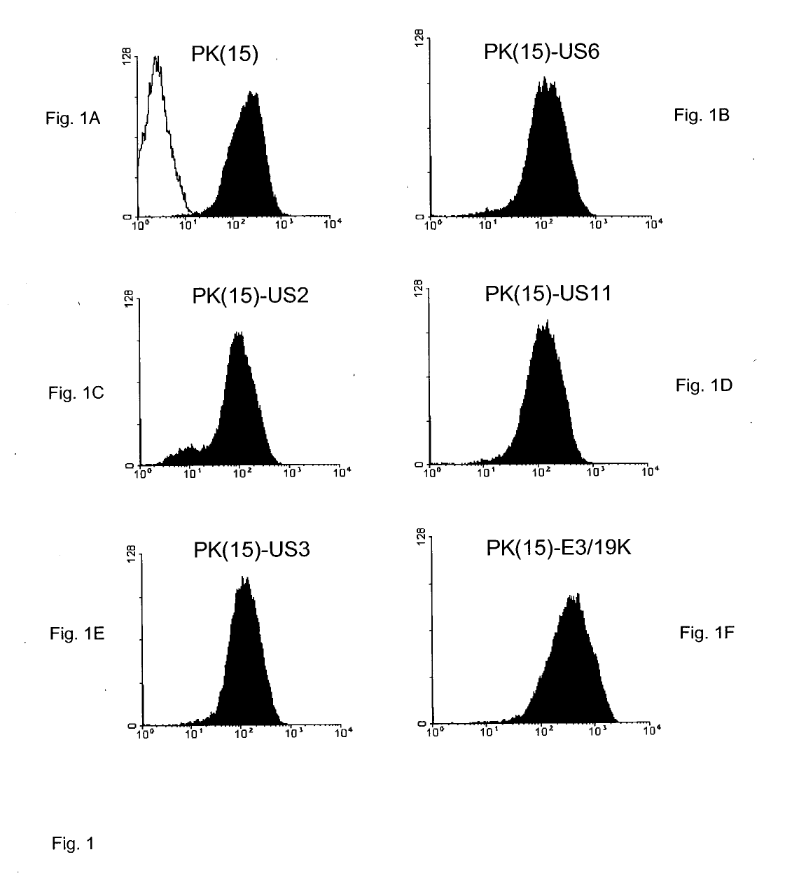

With reference to FIGS. 1-6, the preferred embodiment of the present invention may be described.

The invention describes the use of viral stealth technology for xenotransplantation. The present invention demonstrates three important items. First, a reduction of TAP function by genetic means. Second, that ICP47 expression in pig cells results in a reduction in porcine cell-surface MHC class I. Finally, down-regulation of porcine MHC class I affects human T cell recognition of pig cells.

Six human virus-derived genes which when expressed lead to inhibition of human MHC (HLA) cell-surface expression were tested. None of the four HCMV genes, US2, US3, US6, and US11, affected SLA class I cell-surface expression. Human adenovirus E3/19K was likewise ineffective. HCMV US2, US3, and US11 and adenovirus E3/19K interact with HLA class I heavy chains. The inability of these proteins to affect SLA class I cell-surface expression may reflect specific amino acid differences between HLA and SLA class I proteins. Similarly the failure of HCMV US6 protein, which associates with human TAP, to reduce cell-surface class I expression in porcine cells might be attributed to differences in the amino acid sequences of human and pig TAP proteins. These results are consistent with the species-specificity related to the function of some HCMV gene products in that the HCMV proteins which affect HLA class I expression do not necessarily inhibit mouse MHC (H-2) class I cell-surface expression (Machold R P et al., J Exp Med; 1997; 185: 363; Beersma M F et al., J Immunol 1993; 151: 4455).

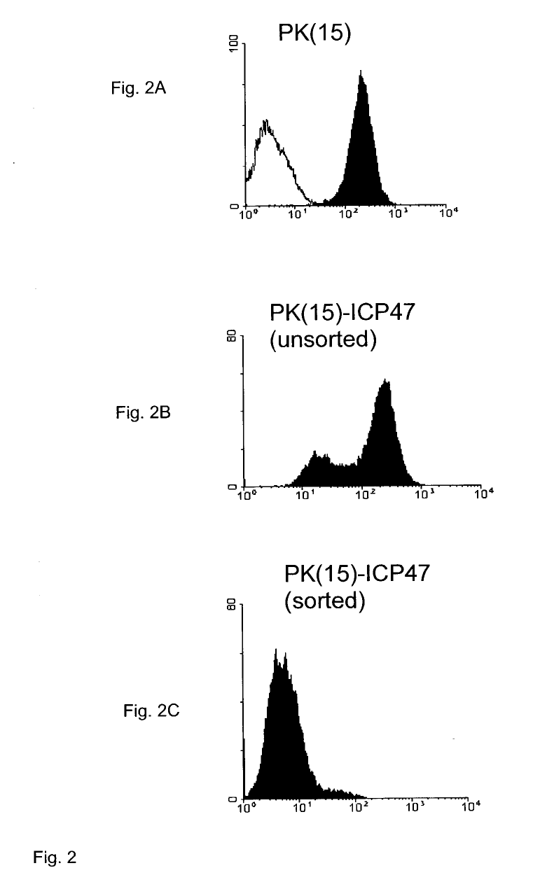

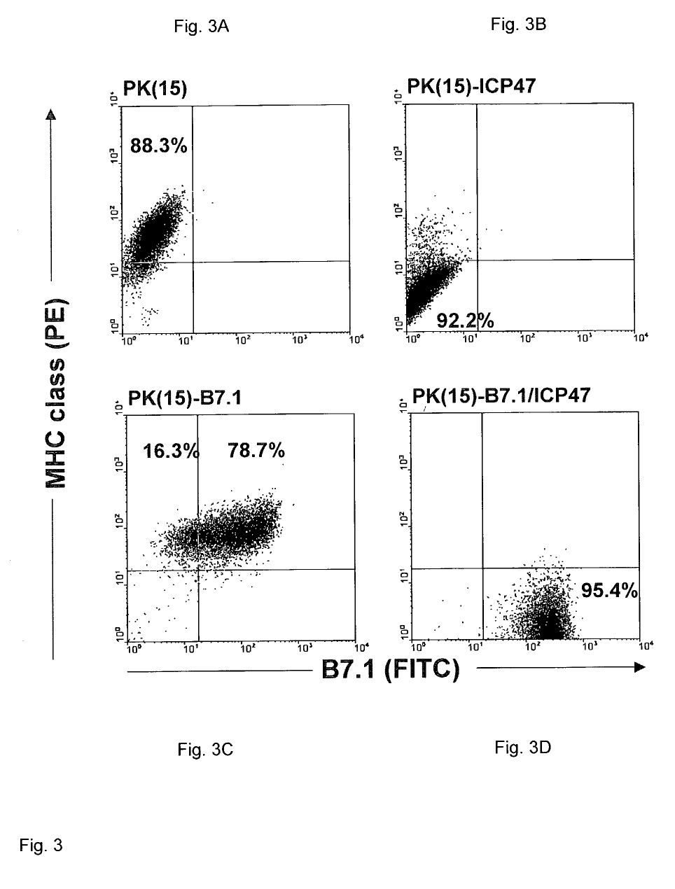

The invention demonstrates that at least one viral stealth gene from a human virus (HSV type II ICP47) was capable of down regulating pig MHC class I cell-surface expression (FIGS. 2 and 3). While HCMV US6 inhibits peptide translocation by TAP, ICP47 inhibits peptide binding to TAP (Tomazin R, et al., EMBO J 1996; 15: 3256). Evidently human and porcine TAP proteins are sufficiently similar in regions responsible for peptide binding (Jugovic et al., J Virol 1998; 72: 5076).

Pig cells, specifically PK(15) cells, expressing ICP47 (and therefore class I-deficient) appear unable to stimulate CD8+ human T cells and seem to be more resistant to human CD8+ T cell-mediated killing.

Effect of human CMV and adenovirus genes on pig kidney MHC class I cell-surface expression. Human CMV US2, US3, US6, and US11 and adenovirus E3/19K genes encode proteins which inhibit HLA class I cell-surface expression by several different mechanisms (Ahn K, et al., Immunity 1997; 6: 613; Fruh K et al., Nature 1995; 375: 415; Hill A et al., Nature 1995; 375: 411; Wiertz E J et al., Cell 1996; 84: 769; Jones T R et al., Proc Natl Acad Sci USA. 1996; 93: 11327; Bennett E M et al, J. Immunol 1999; 62: 5049). To determine if these genes would reduce cell-surface MHC class I levels on pig cells, each gene was cloned into an expression vector (pcDNA3.1) under control of a constitutive promoter and transfected into PK(15) cells, a pig kidney cell line. Pig MHC class I cell-surface levels on transiently or stably transfected cells were quantified by flow cytometry using the PT85A mAb which recognizes a monomorphic determinant on SLA (pig MHC) class I proteins (Tomazin R et al., EMBO J 1996; 15: 3256).

FIG. 1 is typical of the results obtained using human CMV- and adenovirus-derived genes. In short, HCMV US2, US3, US6, US11, and human adenovirus E3/19K failed to substantially reduce pig MHC class I cell-surface levels on PK(15) cells. This was not because of poor transient transfection efficiencies as indicated by co-transfection with a vector encoding green fluorescent protein. Nor did it appear that in stable transfections the CMV and adenovirus gene products were toxic since virtually identical numbers of G-418 resistant colonies were observed in stable transfections with vector alone. Moreover, the CMV and adenovirus gene products did affect HLA class I expression: (1) transient transfection of Hela cells resulted in reduced levels of HLA class I cell-surface levels (indicated by flow cytometry using mAb w6/32 which binds to all HLA class I proteins), and (2) transient transfection of HLA-B27 into PK(15) cells led to measurable levels of HLA-B27 cell-surface expression which was reduced by co-transfected CMV US2, US3, U6, US11, and adenovirus E3/19K.

Down-regulation of pig MHC class I cell-surface levels by HSV ICP47. The HSV type II ICP47 gene product reduces MHC class I cell-surface expression by inhibiting peptide binding by TAP (Tomazin R et al., EMBO J 1996; 15: 3256). Unlike the human CMV and adenovirus genes that were tested, the human HSV ICP47 gene significantly inhibited pig MHC class I cell-surface expression in PK(15) cells (FIG. 2). About 30% of the G-418 resistant cells obtained by stable transfection exhibited an approximately 20-fold reduction in pig MHC class I cell-surface expression. The class I-deficient subset of PK(15) cells could be sorted by FACS to obtain a homogenous population of class I-negative cells (FIGS. 2 and 3).

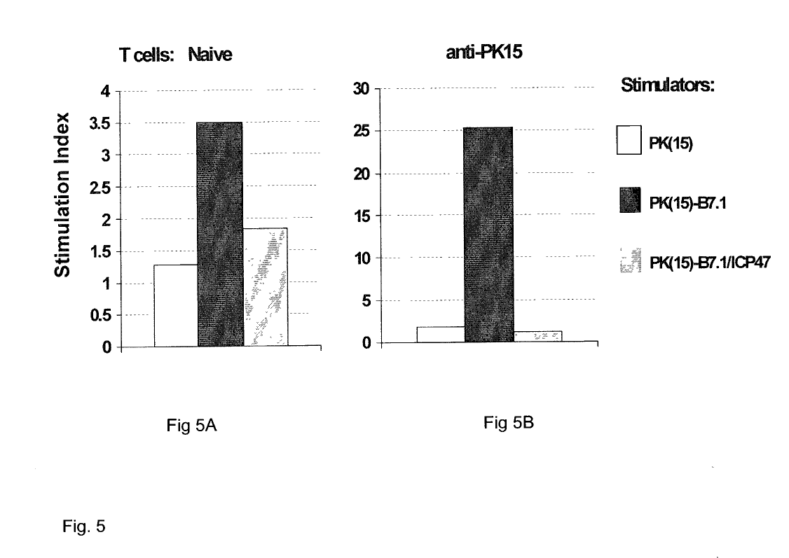

Expression of human B7.1 on PK(15) cells. Preliminary experiments using PK(15) cells to stimulate human T cell proliferation were not successful. We reasoned that this epithelial-derived cell line was ineffective in stimulating human T cell proliferation because PK(15) cells lacked the co-stimulatory molecules B7.1 and B7.2. Importantly in this regard, the interaction of human CD28 with porcine B7.1 seems to be an important component of xenogeneic T cell responses (Lee R S et al., J Immunol 2000; 164: 3434). Thus in order to evaluate the biological consequences (i.e. altered human T cell recognition) of down regulating pig MHC class I by HSV ICP47 on PK(15) cells, we generated stable PK(15) transfectants which constitutively express human B7.1 (FIG. 3). In addition, these cells [PK(15)-B7.1 cells] were then stably transfected with HSV ICP47 to create the PK(15)-B7.1/ICP47 cell line (FIG. 3). As shown below (FIG. 5), the expression of B7.1 appeared essential to generate xenoreactive [(PK(15)-reactive ] T cells.

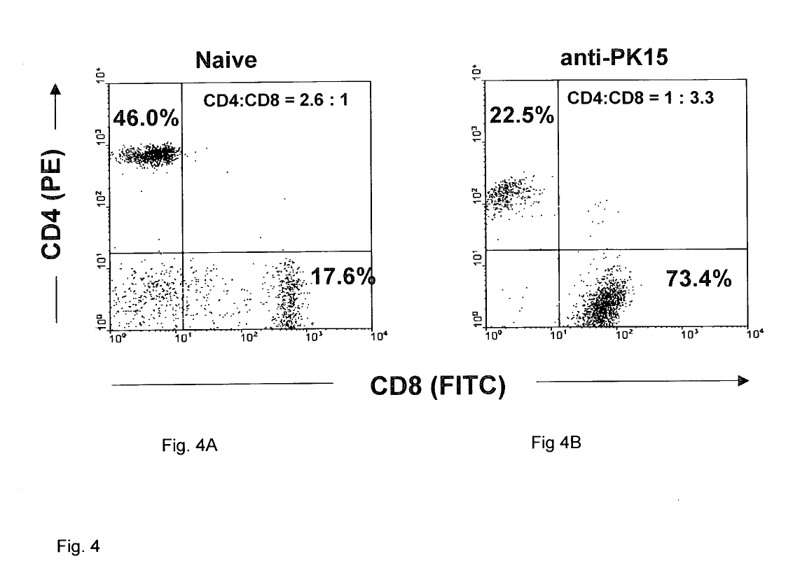

Human CD8+ T cell proliferative response to PK(15)-B7.1 cells. Repeated stimulation (every 4-5 days for three weeks) of human PBMCs with irradiated PK(15)-B7.1 resulted in a visually apparent increase in human lymphocytes by the third stimulation. FACS analysis of expanded lymphocytes, termed anti-PK15 T cells, demonstrated a marked enrichment of CD8+ T cells (FIG. 4). The ratio of CD4:CD8 cells shifted from 2.6:1 in a naïve lymphocyte population to 1:3.3 following repeated stimulation with PK(15)-B7.1 cells. In addition, the levels of CD4 and CD8 decreased approximately 10-fold highly, suggestive of T cell activation. This invention suggests that antigen presentation is primarily occurring via MHC class I molecules in this cell culture system (versus indirect xenoantigen presentation through class II proteins on human APCs present). The present invention, establishes the utility of pig epithelial cells expressing B7.1 to generate xenoreactive human T cells.

Decreased human T cell proliferative response to pig cells expressing ICP47. Both naïve lymphocytes and the xenoreactive (anti-PK15) T cells shown in FIG. 4 were used in T cell proliferation assays using PK(15), PK(15)-B7.1, and PK(15)-B7.1/ICP47 cells (FIG. 3) as stimulators. The results (shown in FIG. 5) support two important conclusions. First, the cell-surface expression of B7.1 was absolutely required for human T cell proliferation in response to pig cells—the proliferation of anti-PK15 T cells in response to untransfected PK(15) cells was negligible . Second, and most important, the reduction of pig cell-surface MHC class I by ICP47 almost completely abolished the human T cell proliferative response to PK(15) cells even though these cells had ample cell-surface expression of B7.1 (FIG. 3). The lack of T cell proliferation induced by class I-deficient pig cells indicates that the human T cells, anti-PK15 cells, directly recognize SLA class I proteins.

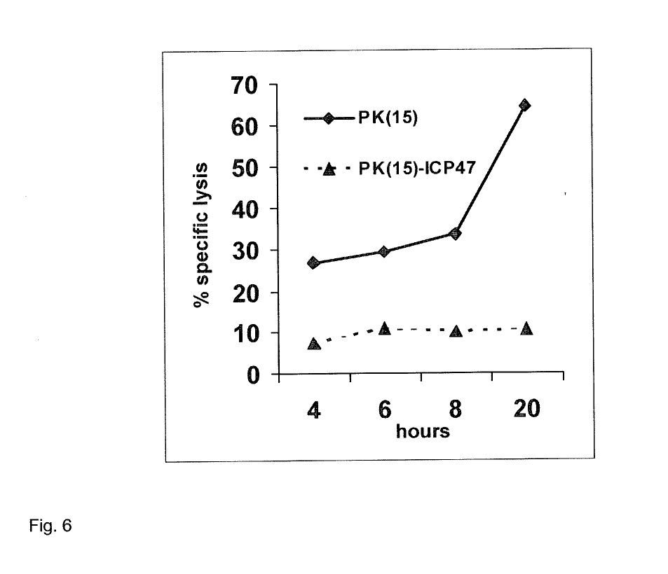

Human T cell-mediated cytotoxicity to pig cells expressing ICP47. Standard cytotoxicity (⁵¹Cr release) assays were performed to determine whether ICP47-mediated down regulation of pig cell-surface expression would impart decreased susceptibility to T cell-mediated lysis. The xenoreactive (PK(15)-reactive) T cell line shown in FIG. 4 was used as effectors with PK(15) and PK(15)-ICP47 cells as targets. Similar to the diminished proliferative response to PK(15)-B7.1/ICP47 cells, PK(15)-ICP47 cells were significantly more resistant to human T cell-medicated lysis than untransfected PK(15) cells (FIG. 6). Thus both T cell proliferation and cytotoxicity studies (FIGS. 5 and 6) strongly suggest that viral stealth proteins may have general utility in reducing T cell-mediated destruction of xenografts.

### EXAMPLES

### Example 1

Cell Lines and Antibodies.

The pig kidney cell line PK(15) (Pirtle E C and Woods L K, Am J Vet Res 1968; 29: 153) was obtained from the American Type Culture Collection (Rockville, Md. USA) and maintained in RPMI 1640 supplemented with 10% fetal calf serum, 100 μg/ml penicillin G, and 100 μg/ml streptomycin sulfate (RPMI/10%). The mAb PT85A which recognizes a monomorphic determinant of porcine MHC class I proteins (Davis W C et al., Vet Immunol Immunopathol 1987; 15: 337) was purchased from VMRD, Inc. (Pullman, Wash. USA). In some flow cytometric analyses, mAb UPC10 was used as isotype control (IgG2a, kappa) and PE-conjugated goat anti-mouse IgG was employed as a secondary antibody; both were purchased from Sigma (St. Louis, Mo., USA). FITC-conjugated anti-human CD80 (B7.1) was purchased from BD Pharmingen (San Diego, Calif. USA). PE-conjugated anti-human CD4 and FITC-conjugated anti-human CD8 mAbs were purchased from CalTag Laboratories (Burlingame, Calif. USA).

### Example 2

Cloning of Viral Genes and Human B7.1 cDNA.

Adenovirus E3/19K, HCMV US2, US3, US6, and US11, and human HSV type II ICP47 genes were all cloned by PCR and ligation into pcDNA3.1 (Stratagene, Torrey Pines, Calif. USA). All primers used in PCR amplification included unique restriction sites to facilitate cloning. The 5′ primer in each case contained sequences encompassing the initiating methionine codon and the 3′ primer in each instance overlapped sequences corresponding to the termination codon of each gene. Sequences inserted into the multiple cloning site of pcDNA3.1 are expressed under control of the CMV I-E (immediate early) promoter.

For cloning of adenovirus E3/19K, Adenovirus 2 DNA was used as template for PCR using the primers E3-5′ (CGAATTCAACATCCAAGATGAAGGTAC) and E3-3′ (CGGAATTCTCAGTGATGGTGATGGTGATGAGGCATTTTCTTTCATC) which includes sequences encoding six histidines immediately preceding the termination codon. The E3/19K PCR product was digested with EcoRI and ligated into EcoRI-cleaved pcDNA3.1. Restriction enzyme mapping identified a clone with the E3/19K gene in proper orientation.

PCR-mediated cloning of HCMV US2, US3, US6, and US11 genes utilized human CMV (Towne strain) DNA as template and the following primers:

US2-5′
(GCGGATCCACACGCTGTTTCACCATG),
US2-3′
(GCGAATTCCCGGGCGTCTCAGCACACG),
US3-5′
(GCGGATCCTTCGGAGCCATGAAGCCGGTG),
US3-3′
(GCGGAATTCGTACCTGTTAAATAAATCG),
US6-5′
(GCGGATCCTTCACTATGGATCTCTTG),
US6-3′
(CGAATTCATCAGGAGCCACAACGTCGAATC),
US11-5′
(GCGGATCCTTGTAAGACAGAATGAACC), and
US11-3′
(GCGAATTCAGTTCTATATATCACCACTG).

PCR products were digested with EcoRI and BamHI and ligated into EcoRI- and BamHI-cut pcDNA3.1.

HSV II ICP47 was cloned using total cellular DNA from cells infected with HSV II as template for PCR using ICP47-5′ (CCGAATTCGAGATCGTATCAAGGGGCC) and ICP47-3′ (CCGGATCCGGGACACCATGTCTTGGG) as primers. ICP47 PCR products were digested with EcoRI and BamHI and ligated into EcoRI- and BamHI-cut pcDNA3.1.

Cloning of human B7.1 (CD80) was accomplished via PCR using B7.1-5′ (CTAAGCTTCTGAAGCCATGGGCCAC) and B7.1-3′ (GGCTCGAGCTGCGGACACTGTTATACAGG) as primers and an aliquot of a human kidney cDNA library (purchased from ClonTech, Palo Alto, Calif. USA) as template. B7.1 PCR products were digested with HindIII and Xhol and ligated into HindIII- and Xhol-cleaved pcDNA3.1.

All clones were completely sequenced to verify correct orientation and open reading frames. The CMV Towne strain US2, US3, US6, and US11 genes differed only slightly (<3% at the nucleotide level) from the known sequence of the corresponding genes of the CMV AD169 strain. All HCMV Towne strain genes were predicted to encode proteins of near identical size to those encoded by the AD169 strain. GenBank Accession numbers of HCMV (Towne) US2, US3, US6, and US11 are AY072773, AY072774, AY072775, and AY072776, respectively.

### Example 3

Stable Transfection of PK(15) Cells.

PK(15) cells were transfected by electroporation. In brief, 2×10⁶ PK(15) cells in 200 μl RPMI/10% were mixed with 20 μg plasmid DNA (in 200 μl RPMI/10%) in an electroporation cuvette. Following electroporation (250 V, 960 μF), cells were plated in 10 ml RPMI/10% and two days later the media was changed to RPMI/10% containing 500 μg/ml G-418. pcDNA3.1 contains the neo gene under control of the SV40 early promoter. Media was changed every 4 to five days and G-418 resistant colonies were visible after about two weeks. To generate PK(15)-B7.1/ICP47 cells, PK(15) cells were first transfected with B7.1 in pcDNA3.1 and selection employed G-418. The resulting PK(15)-B7.1 cells were then co-transfected with 20 μg ICP47 (in pcDNA3.1) and 1 μg pPUR (ClonTech, Palo Alto, Calif. USA) which confers puromycin resistance. Two days after electroporation, cells were grown in RPMI/10% with 500 μg/ml G-418 and 2 μg/ml puromycin. In this application, PK(15) transfectants are designated by the gene(s) they have been transfected with; e.g. PK(15)-B7.1/ICP47 cells express both human B7.1 and HSV II ICP47.

### Example 4

Flow Cytometric Analyses.

PK(15) cells, removed from plates by trypsinization, or human lymphocytes were washed once with wash buffer (phosphate buffered saline, PBS, with 2% fetal calf serum and 0.2% NaN3) and incubated on ice for 30-60 minutes with saturating concentrations of primary antibody. Cells were washed twice with wash buffer to remove unbound antibody. When PT85A was used as primary antibody, the cells were subsequently incubated with PE-conjugated goat anti-mouse IgG for 30-60 minutes on ice in wash buffer. Prior to flow cytometry all cells were fixed in PBS containing 1% paraformaldehyde. Flow cytometric analyses were performed using the FACSCalibur instrument (Becton Dickinson, Franklin Lakes, N.J. USA).

### Example 5

Human Iymphocyte Preparation and Expansion.

Peripheral blood mononuclear cells were obtained from whole blood by Ficoll-Hypaque density gradient centrifugation. Monocytes were removed by adherence to plastic in a two hour incubation at 37 C. The resulting lymphocyte populations were immediately expanded using PK(15)-B7.1 cells as stimulators. For T cell expansion, confluent monolayers of PK(15)-B7.1 cells in T25 (25 cm²) flasks (about 3-4×10⁶ cells) were gamma irradiated at the minimum radiation dose (30,000 rads) that was empirically determined to completely halt PK(15) cell division. Following irradiation, the PK(15) monolayers were washed with PBS and 10 ml of RPMI/10% with 10 U/ml IL-2 were added with lymphocytes at a final concentration of 106/ml. T cell expansion continued for 3-4 weeks with restimulation every 4-5 days. The resulting T cell lines are designated as “anti-PK15 T cells” in this application.

### Example 6

T Cell Proliferation Assays.

T cell proliferation was quantified by ³H-thymidine incorporation. PK(15), PK(15)-B7.1 and PK(15)-B7.1/ICP47 cells were used as stimulators after they had been gamma irradiated (30,000 rads). Effector cells were either naïve lymphocytes (never having been stimulated with porcine cells) or T cells that had undergone repeated stimulation with PK(15)-B7.1 cells (anti-PK15 T cells) as described above. Proliferation assays were carried out in triplicate in 96-well flat bottom dishes using 10⁵ anti-PK15 T cells/well at an effector:stimulator ratio of 10:1. Following a two day incubation at 37 C., ³H-thymidine was added at 1 μCi/well and incubation continued for another two days. Cells were harvested on glass fiber filter papers and radioactivity was counted using a Packard Matrix96 direct beta counter. The stimulation index was calculated by the formula:

cpm(experimental)−cpm(stimulator alone)/cpm(effector alone)×100.

### Example 7

T Cell Cytotoxicity Assays.

T cell cytotoxicity was measured by standard ⁵¹Cr release assays. Effector cells were anti-PK15 T cells generated as described above. Confluent monolayers of target cells, PK(15) or PK(15)-ICP47 cells, were incubated in RPMI/10% with 10 μCi/ml ⁵¹Cr for 16 hours at 37 C. The monolayers were washed three times with PBS prior to trypsinization. Cytotoxicity assays were performed in triplicate in 96 well U-bottom dishes using 104 target cells/well at an effector:target ratio of 10:1 in a final volume of 200 μl. After various times of incubation at 37 C. (4, 6, 8, and 20 hours), 25 μl of supernatant was removed and the radioactivity counted using a Packard gamma counter. Percent specific lysis was calculated using the formula:

cpm(experimental)−cpm(spontaneous)/cpm(maximum)−cpm(spontaneous)×100

Sequence Listing

The nucleic and amino acid sequences listed in the accompanying sequence listing are shown using standard letter abbreviations for nucleotide bases. Only one strand of each nucleic acid sequence is shown, but the complementary strand is understood as included by any reference to the displayed strand.

SEQ ID NO. 1 shows the PCR primer used for cloning the human adenovirus type 3 E3/19k gene.

SEQ ID NO. 2 shows the PCR primer used for cloning the human adenovirus type 3 E3/19k gene.

SEQ ID NO. 3 shows the PCR primer used for cloning the human cytomegalovirus US2 gene.

SEQ ID NO. 4 shows the PCR primer used for cloning the human cytomegalovirus US2 gene.

SEQ ID NO. 5 shows the PCR primer used for cloning the human cytomegalovirus US3 gene.

SEQ ID NO. 6 shows the PCR primer used for cloning the human cytomegalovirus US3 gene.

SEQ ID NO. 7 shows the PCR primer used for cloning the human cytomegalovirus US6 gene.

SEQ ID NO. 8 shows the PCR primer used for cloning the human cytomegalovirus US6 gene.

SEQ ID NO. 9 shows the PCR primer used for cloning the human cytomegalovirus US11 gene.

SEQ ID NO. 10 shows the PCR primer used for cloning the human cytomegalovirus US11 gene.

SEQ ID NO. 11 shows the PCR primer used for cloning the human herpesvirus 2 ICP47 gene.

SEQ ID NO. 12 shows the PCR primer used for cloning the human herpesvirus 2 ICP47 gene.

SEQ ID NO. 13 shows the PCR primer used for cloning the human B7.1 gene.

SEQ ID NO. 14 shows the PCR primer used for cloning the human B7.1 gene.

SEQ ID NO. 15 shows the nucleotide sequence of the human cytomegalovirus, Towne strain, US2 gene, Accession No. AY072773.

SEQ ID NO. 16 shows the nucleotide sequence of the human cytomegalovirus, Towne strain, US3 gene, Accession No. AY072774.

SEQ ID NO. 17 shows the nucleotide sequence of the human cytomegalovirus, Towne strain, US6 gene, Accession No. AY072775.

SEQ ID NO. 18 shows the nucleotide sequence of the human cytomegalovirus, Towne strain, US11 gene, Accession No. AY072776.

SEQ ID NO. 19 shows the amino acid sequence of the human cytomegalovirus, Towne strain, US2 protein, Accession No. AA67141.

SEQ ID NO. 20 shows the amino acid sequence of the human cytomegalovirus, Towne strain, US3 protein, Accession No. AYO72774.

SEQ ID NO. 21 shows the amino acid sequence of the human cytomegalovirus, Towne strain, US6 protein, Accession No. AY072775.

SEQ ID NO. 22 shows the amino acid sequence of the human cytomegalovirus, Towne strain, US11 protein, Accession No. AAL67144.

What is claimed is:

1. An isolated or synthetic protein comprising an amino acid sequence selected from the group consisting of:
(a) the amino acid sequences shown in SEQ ID NO. 19, 20, 21, and 22;
(b) amino acid sequences that differ from those specified in (a) by at least one conservative amino acid substitutions that retain biological activity; and
(c) fragments of an amino acid sequence shown in SEQ ID NO. 19, 20, 21, and 22 that retain biological activity.

2. An isolated nucleic acid molecule having a nucleotide sequence selected from the group consisting of:
(a) the nucleotide sequences shown in SEQ ID NO. 15, 16, 17, and 18;
(b) a complementary strand of a nucleotide sequence shown in SEQ ID NO. 15, 16, 17, and 18; and
(c) fragments of a nucleotide sequence shown in SEQ ID NO. 15, 16, 17, and 18.

3. A recombinant vector including a nucleic acid molecule according to claim 2 and regulatory elements necessary for expression of the nucleic acid in a cell.

4. A transgenic cell transformed with said recombinant vector according to claim 3.

5. A transgenic organism having said transgenic cells of claim 4.

6. A method of generating xenoreactive human T cells, comprising introducing into a nonhuman cell, a sequence selected from the group consisting of:
(a) the amino acid sequences shown in SEQ ID NO. 19, 20, 21, and 22;
(b) human B7.1.

7. The method of claim 6 wherein the said nonhuman cell is a porcine cell.

8. The method of claim 7 wherein said porcine cell is a pig kidney cell.

9. The method of claim 7 wherein said porcine cell is a pig pancreatic islet cell.

10. The method of claim 7 wherein said porcine cell is a pig neuronal cell.

11. The method of claim 7 wherein said porcine cell is a pig heart cell.

12. The method of claim 7 wherein said porcine cell is a pig liver cell.

13. The method of claim 7 wherein said porcine cell is a pig lung cell.

14. The method of claim 7 wherein said porcine cell is a pig skin cell.

15. A method of inhibiting recognition of a nonhuman cell after xenotransplantation, comprising introducing into said nonhuman cell, a sequence selected from the group consisting of:
(a) the amino acid sequences shown in SEQ ID NO. 19, 20, 21, and 22;
(b) human B7.1, and;
(c) herpes simplex virus ICP47 protein.

16. The method of claim 15 wherein the said nonhuman cell is a porcine cell.

17. The method of claim 16 wherein said porcine cell is a pig kidney cell.

18. The method of claim 16 wherein said porcine cell is a pig pancreatic islet cell.

19. The method of claim 16 wherein said porcine cell is a pig neuronal cell.

20. The method of claim 16 wherein said porcine cell is a pig heart cell.

21. The method of claim 16 wherein said porcine cell is a pig liver cell.

22. The method of claim 16 wherein said porcine cell is a pig lung cell.

23. The method of claim 16 wherein said porcine cell is a pig skin cell.

24. A method of conferring resistance of a nonhuman cell after xenotransplantation, comprising introducing into said nonhuman cell, a sequence selected from the group consisting of:
(a) the amino acid sequences shown in SEQ ID NO.19, 20, 21, and 22;
(b) human B7.1, and;
(c) herpes simplex virus ICP47 protein.

25. The method of claim 24 wherein the said nonhuman cell is a porcine cell.

26. The method of claim 25 wherein said porcine cell is a pig kidney cell.

27. The method of claim 25 wherein said porcine cell is a pig pancreatic islet cell.

28. The method of claim 25 wherein said porcine cell is a pig neuronal cell.

29. The method of claim 25 wherein said porcine cell is a pig heart cell.

30. The method of claim 25 wherein said porcine cell is a pig liver cell.

31. The method of claim 25 wherein said porcine cell is a pig lung cell.

32. The method of claim 25 wherein said porcine cell is a pig skin cell.

33. A method of inhibiting human T cell proliferative response to a nonhuman cell, after xenotransplantation, comprising introducing into said nonhuman cell, a sequence selected from the group consisting of:
(a) the amino acid sequences shown in SEQ ID NO.19, 20, 21, and 22;
(b) human B7.1, and;
(c) herpes simplex virus ICP47 protein.

34. The method of claim 33 wherein the said nonhuman cell is a porcine cell.

35. The method of claim 34 wherein said porcine cell is a pig kidney cell.

36. The method of claim 34 wherein said porcine cell is a pig pancreatic islet cell.

37. The method of claim 34 wherein said porcine cell is a pig neuronal cell.

38. The method of claim 34 wherein said porcine cell is a pig heart cell.

39. The method of claim 34 wherein said porcine cell is a pig liver cell.

40. The method of claim 34 wherein said porcine cell is a pig lung cell.

41. The method of claim 34 wherein said porcine cell is a pig skin cell.
